(19) United States
(12) Patent Application Publication
(10) Pub. No.: US 2015/0164697 A1
(43) Pub. Date: Jun. 18, 2015

(54) STABILIZER, BARRIER DISC AND WOUND DRESSING COMPRISING STABILIZER, METHOD FOR CONTROLLING THE POSITION OF A WOUND DRESSING OR BARRIER DISC, AND METHOD FOR FACILITATING DRAINAGE FROM A WOUND DRESSING OR BARRIER DISC IN NEGATIVE PRESSURE WOUND TREATMENT

(71) Applicants: Malin Malmsjö, Lomma (SE); Richard Ingemansson, Malmö (SE); Sandra Lindstedt Ingemansson, Malmö (SE); Ola Rickardsson, Malmö (SE)

(72) Inventors: Malin Malmsjö, Lomma (SE); Richard Ingemansson, Malmö (SE); Sandra Lindstedt Ingemansson, Malmö (SE); Ola Rickardsson, Malmö (SE)

(21) Appl. No.: 14/232,473

(22) PCT Filed: Jul. 6, 2012
(86) PCT No.: PCT/SE2012/050804

§ 371 Date: Feb. 3, 2014

(30) Foreign Application Priority Data
Jul. 19, 2011 (SE) 1100547-7
Oct. 4, 2011 (SE) 1150912-2

(51) Int. Cl. A61F 13/00; A61M 1/00

(52) U.S. Cl. CPC A61F 13/00068; A61M 1/0088

(57) ABSTRACT

A stabilizer for stabilization of a position of a wound dressing or barrier disc (11) for use in negative pressure wound therapy, NPWT, or for stabilization of a barrier disc or wound dressing and/or maintenance of a drainage capacity of a wound dressing for use in NPWT is arranged to protrude from a surface of the wound dressing or barrier disc (11). A barrier disc (11) and a wound dressing comprising such a stabilizer are also disclosed, as well as a method for controlling the position of a wound dressing or barrier disc (11) in NPWT, and a method for maintaining or facilitating drainage from a wound dressing or barrier disc in NPWT.

### TECHNICAL FIELD OF THE INVENTION

The present invention relates to a stabilizer for stabilization and positioning of a soft wound dressing or barrier disc during negative pressure wound therapy (NPWT). The present invention further relates to a barrier disc and a wound dressing, to a method for controlling the position of a wound dressing or barrier disc and to a method for facilitating drainage from a wound dressing or barrier disc.

### BACKGROUND ART

Wound drainage has been used since the 1960's to aid in a speedy recovery of surgical patients, primarily after gastrointestinal incisions, but has later been developed for a range of situations, such as cerebral drainage, orthopedic drainage and sternum drainage. Without the use of an effective drainage system, a post-surgical wound can easily become infected. These infections can become extremely severe and spread to other areas and organs of the body. The most common three wound drainage types are Penrose drains, Jackson-Pratt drains and negative pressure drains.

The Penrose drain is basically a length of soft rubber or silicon tubing that is placed inside of a wound area to facilitate the drainage of wound areas. Hydrocephalus patients have found this drainage format beneficial in draining cerebrospinal fluid.

The Jackson-Pratt drain, or Bulb drain, applies a continuous suction pressure to the wound by use of a flexible bulb. The bulb is used both as a mechanism of providing suction and as a reservoir for the escaping fluid.

In 1997, Morykwas and Argenta published three landmark articles regarding experience with a “new method for wound control and treatment.” A system was described where subatmospheric pressure was applied through a closed system to an open wound for periods of 48 hours. Subatmospheric pressure was directed at the surface of the wound through an interface between the wound surface and a polyurethane sponge to allow for distribution of the negative pressure using either a constant or intermittent mode based on clinical experience.

Negative pressure wound therapy is thought to promote wound healing through multiple actions—it creates a moist wound healing environment, drains exudate, reduces tissue edema, contracts the wound edges and mechanically stimulates the wound bed, and influences the blood perfusion of the wound edge, leading to angiogenesis and the formation of granulation tissue.

The definition of Negative Pressure Wound Therapy varies but independent definitions centre around negative pressure in the wound bed. Thus it has been defined as:

“Negative Pressure Therapy is the application of subatmospheric pressure either continuously or intermittently to an open wound” or

“Negative Pressure Wound Therapy is a non-invasive treatment by which controlled localized negative pressure is delivered to a wide variety of acute, sub-acute and chronic wounds”.

Negative pressure wound therapy (NPWT) is a topical treatment intended to promote healing in acute and chronic wounds. It involves the application of negative pressure (suction) to the wound bed.

NPWT involves application of a non-adherent, porous wound dressing, a drainage tube placed adjacent to or inserted in the dressing, an occlusive transparent film sealing the wound and the drainage tube, and a connection to a vacuum source, which supplies the negative pressure. The concept is to turn an open wound into a controlled, closed wound while removing excess fluid from the wound bed through suction forces, thus enhancing circulation and disposal of cellular waste from the lymphatic system.

This technique is usually considered for hard-to-heal wounds, e.g., chronic wounds (those that fail to progress through the normal phases of healing—inflammation, proliferation, maturation—and thus do not heal), acute wounds (wounds that are expected to heal and demonstrate evidence of progression through the phases of healing), and difficult wounds (wounds with such associated factors as diabetes, arterial insufficiency, and venous insufficiency). Common applications for NPWT are:
Acute wounds
Sternotomy/mediastinitis
Wounds infected after vascular surgery
Infected abdomen
Partial- and full-thickness burns
Surgically created wounds and surgical dehiscence*
Neuropathic (diabetic) wounds
Venous or arterial insufficiency ulcer unresponsive to standard therapy
Traumatic wounds (i.e., flap or meshed graft)
Pressure ulcers (stage 3 or 4)
Patients with other medical problems; i.e., diabetes, coronary artery disease, or renal disease, may be more susceptible to wound dehiscence and delayed wound healing. NPWT helps resolve the problem with healing in these hard-to-heal wounds.

In cardiac surgery, e.g., by-pass operation of the heart, the sternum is cut lengthwise, and quite often the left pleura is opened as well. This generates a so called sternotomy wound. Following surgery, the sternotomy wound is closed with sternal wires and left to heal.

In a number of patients, sternal wound infections occur. These may be superficial, involving only soft tissues, or deep, where the sternal bone itself is infected. Deep sternal wound infection (DSWI) is also called poststernotomy mediastinitis, and occurs in about 1 to 5% of those undergoing cardiac surgery by sternotomy. Such poststernotomy mediastinitis occurs particularly in a risk group of patients, such as those suffering from diabetes mellitus, low left ventricular ejection fraction, obesity, renal failure, and three-vessel disease. Established treatment of poststernotomy mediastinitis includes debridement with frequent postoperative irrigation, change of wound dressings and direct secondary closure or secondary closure by use of vascularized muscle flaps. The reported early mortality using these established techniques in poststernotomy mediastinitis following coronary bypass surgery is between 8 and 25%.

However, the introduction of a technique for using negative pressure wound therapy (NPWT) to treat poststernotomy mediastinitis has essentially reduced the mortality due to mediastinitis (Sjogren, J., et al. Ann Thorac Surg. 80: 1270, 2005). The NPWT technique entails applying negative pressure to a wound in a controlled manner. A wound filler dressing in the form of a sterile polyurethane foam is placed between the sternal edges (most commonly in the form of a sterile polyurethane foam), but not below the level of the sternum, in order not to affect hemodynamic and respiratory function. A second layer of wound filler is often placed subcutaneously and secured with a running suture to the surrounding skin. This facilitates the application of the adhesive drape. Drainage tubes are inserted into the foam. The wound is then sealed with a transparent adhesive drape. The drainage tubes are connected to a purpose-built vacuum pump and a canister for collection of effluents. Initially, a low pressure (e.g. −50 mmHg) is applied to allow adjustment of the foam as the air is evacuated. If the wound geometry and foam contraction are considered satisfactory, common pressures that are applied are −75 mmHg to −125 mmHg. Air leakage is known to dry out the wound and can be prevented by additional draping. Most of the patients can be extubated and mobilized immediately after NPWT application. Revisions and dressing changes are performed regularly, e.g. three times a week, under aseptic conditions and general anaesthesia. The sternal wound can be closed and mostly rewired when the infection has resolved, typically after 1-3 weeks of NPWT treatment. The method is simple and effective and is believed to combine the benefits of closed and open wound treatment to create an environment that promotes wound healing.

However, a very serious potential complication of NPWT therapy of sternotomy wounds is the risk of serious damage to the heart and surrounding structures, in particular the risk of right ventricle disruption (RVD) of the heart when the heart is pulled towards the sharp edge of the divided sterna bone. Khoynezhad et. al. (2004) reviewed 3 cases of their own alongside literature reports of 39 earlier cases. These 3 cases arose out of 40 patients with mediastinitis (7.5%). If damage is severe these events can be fatal. The authors considered that right ventricle-tissue adhesions to the infected sternal bone and soft tissues were a likely causative factor, but clearly the presence and mobility of the un-stabilized cut sternal bone offers a significant risk in the event of patient breathing or coughing.

There have been a number of reports of right ventricular rupture in the literature. Sartipy et. al. (2006) describe 5 NPWT cases from Sweden, 3 of which were fatal. In a series of 21 DSWI treated with NPWT Bapat et. al, (2008) report one RVD death out of 5 total mortalities. Ennker et. al, (2009) also describe one RVD fatality out of 54 DSWI patients treated with NPWT. Placement of several layers of paraffin gauze (up to six layers) between the heart and the foam are recommended by Sjogren et. al., (2006) to provide protection between the heart and the sternum. Sartipy et. al. (2006) speculate that inadequate placement of the paraffin gauze protective layers may have influenced the occurrence of RVD. Inadequate stabilization by using too low or ineffective negative pressure might also lead to sternal mobility and the risk of RVD (Malmsjö et. al., 2007). Malmsjö et. al., (2009) have recently published an assessment of the heart during NPWT using real-time magnetic resonance imaging in a 70 Kg pig model and the result show during NPWT the heart can be pulled up against the sternal edges.

Taken together, it is established that poststernotomy mediastinitis can be effectively treated using NPWT, but it is a major concern that the method is not completely reliable and can cause heart rupture.

Heart rupture and death is the most devastating complication and occur in 4 to 7% of all cases treated for mediastinitis after cardiac surgery (Khoynezhad et. al. (2004), Sartipy et. al. (2006), Bapat et. al., (2008) and Ennker et. al., (2009)). In November 2009 and February 2011 the US FDA filed two alerts, see below, and the importance of protecting the heart and other exposed organs is emphasized in international scientific literature. FDA has thus issued warning letters reading:

FDA Preliminary Public Health Notification: Serious Complications Associated with Negative Pressure Wound Therapy Systems dated Nov. 13, 2009, wherein FDA issued a warning for complications as damage to organs, such as heart and lungs, being lethal, which signals the fact that the problem with NPWT remains in heart surgery. Similar complications also occur in the NPWT of other organs such as intestines and blood vessels.

FDA Safety Communication: UPDATE on Serious Complications Associated with Negative Pressure Wound Therapy Systems dated Feb. 24, 2011.

FDA warns of organ damage during NPWT in 2009 and highlights the importance of the problem in 2011:

“Extensive bleeding occurred in patients with vascular grafts (such as femoral and femoral-popliteal grafts), in sternal and groin wounds, in patients receiving anti-coagulant therapy.” (2009)

“FDA will work with the manufacturers and health care professional organizations to make important information known to the clinical community. Additionally, FDA continues to work with manufacturers to ensure the development, testing and promulgation of methods for reducing the risk associated with these devices and to minimize the complications from adverse events that may occur in the course of normal usage. If the results of any survey raise serious concerns about the safety of these devices, FDA may convene an Ad Hoc group of clinical and manufacturing representatives to discuss further actions.” (2011)

NPWT of larger abdomen incisions create another serious problem in that the underlying tissues, such as spleen, intestines, liver and others cannot withstand the mechanical power of the suction forces created by NPWT. In the abdomen, there is a risk of damages causing fistulas and adherences which may result in a frozen abdomen and death.

WO 2007/123451 and WO 2009/142598 disclose implantable, disposable barrier discs to be used in the negative pressure treatment of wounds, in particular sternotomy wounds, wherein the barrier disc consists of a rigid material withstanding a pressure of −50 mmHg without causing deformation to the barrier, and wherein the barrier is perforated to allow drainage of wound fluid through said barrier disc.

These barrier discs effectively protect sensitive tissue structures or organs underlying the NPWT, such as the heart at sternotomy, or the abdomen tissues at a major abdomen surgery incision, and blood vessels, when exposed in the wound bed.

A clinical evaluation using the barrier discs disclosed in WO 2007/123451 and WO 2009/142598 has been made and the results have been reported in a number of scientific articles (e.g. Lindstedt et al (2011a); Lindstedt et al (2011 b); Lindstedt et al (2011c); Lindstedt et al (2011 d); Lindstedt et al (2011e); Lindstedt et al (2011e); Lindstedt et al (2012a); Lindstedt et al (2012b); Anesäter et al (2012); Malmsjo et al (2009); Anesäter et al, In press Wound Rep Regen; and Anesäter et al (2011).

The articles on the evaluation further discuss the discovery of the cause of heart rupture in connection with NPWT in the treatment of poststernotomy mediastinitis, and the discovery that these events could not be prevented by placing paraffin gauze over the anterior portion of the heart, the paraffin gauze being used in connection with other wound dressings, foam etc. It was discovered that insertion of a rigid disc between the anterior part of the heart and the inside of the thoracic wall was successful in providing protection. In this connection, the barrier discs disclosed in WO 2007/123451 and WO 2009/142598 were invented. Examples of barrier discs embodying these inventions are marketed by the company Shieldheart MedTech AB under the names Heartshield, Vesselshield, and Gutshield.

As discussed above, the use of barrier discs such as the ones disclosed in WO 2007/123451 and WO 2009/142598 has been shown to be beneficial when treating wounds, such as sternotomy wounds and abdominal wounds, and other wounds with exposed sensitive structures, such as blood vessels, particularly using NPWT.

It has turned out however, that the position of an underlying wound dressing or barrier disc needs to be controlled in an efficient way, as it has been shown occasionally that the disc may move away from the initial site, which in turn may compromise the healing or cause damage to the underlying tissue.

### SUMMARY OF THE INVENTION

It is an object of the present invention to solve or at least lessen the above-mentioned problem.

It is a particular object of the present invention to provide a device that enables stabilization of a wound dressing or barrier disc for use in negative pressure wound therapy (NPWT).

A further object is to provide a wound dressing and/or a barrier disc for use in NPWT which may be more securely held in place in the wound.

Yet another object is to provide a method for controlling the position of a wound dressing or barrier disc in NPWT, which makes it possible to securely control the position of the wound dressing or barrier disc.

Still another object is to provide a method for facilitating drainage from a wound dressing or barrier disc in negative pressure wound treatment. The above-mentioned objects are achieved, in full or at least in part, by a stabilizer for stabilization of a position of a wound dressing or barrier disc for use in negative pressure wound therapy, said stabilizer being arranged to protrude from a surface of the wound dressing or barrier disc.

The stabilizer may also be used for stabilization and/or maintenance of a drainage capacity of a wound dressing for use in negative pressure wound therapy.

To be more exact, the present invention provides a stabilizer for:
i) stabilization of a position of a wound dressing for use in negative pressure wound therapy, and/or
ii) stabilization of a position of a barrier disc for use in negative pressure wound therapy, and/or
iii) stabilization and/or maintenance of a drainage capacity of a wound dressing for use in negative pressure wound therapy,
   said stabilizer being arranged to protrude from a surface of the wound dressing or barrier disc. A stabilizer in accordance with the present invention may provide all three of i), ii) and iii) outlined above or any combination of two of i)-iii) or only one of i)-iii).

When such a stabilizer is arranged on a wound dressing or barrier disc, the position of the wound dressing or barrier disc in the wound can be stabilized and it also enables stabilization or maintenance of the drainage capacity of the wound and wound dressing. Additionally, the position, shape and/or drainage capacity of additional wound dressing materials can be stabilized. Further, placement of the wound dressing or barrier disc is simplified, since the person placing the wound dressing or barrier disc may observe the protruding stabilizer in order to verify the position of the wound dressing or barrier disc on which the stabilizer is arranged. Additionally, placement of the wound dressing or barrier disc is quicker, since the stabilizer may automatically guide the wound dressing or barrier disc into position in the wound. Another advantage of using a stabilizer arranged to protrude from the wound dressing or barrier disc is that the stabilizer may stabilize the edges of the wound. For example, there are multiple problems with instabilzaton of the sternotomy wound due to fractured ribs and/or degradation of the sternum bone following the surgery and/or osteitis, stabilisation of these sternum wound edges is crucial in maintaining the thoracic cavity. Stabilisation of the wound edges may reduce the tensile forces and lacerations of the wound edges, thereby reducing the pain of the patient during NPWT. When used in NPWT of a sternotomy wound, the stabilization of the sternal edges may facilitate the cease of mechanical ventilation of the patient. The pressure used during NPWT may be more evenly distributed if a stabilizer is used, thereby reducing the risk of cut-off areas of the wound, particularly deep in the wound. The stabilizer may also make it possible to use different pressures in different parts of the wound, for instance using one pressure near the surface of the wound and another pressure deep in the wound. This may be done by directing the negative pressure to the desired parts by specific drainage channels that are incorporated in the device. Particularly, the stabilizer may be used during negative pressure wound therapy to provide additional, beneficial effects during the treatment.

In the context of the present application, the term “wound dressing” relates to any wound dressing known to the skilled person. The wound dressing may be placed under and/or around the stabilizer. It may also be a combination of a rigid disc and a softer wound dressing.

The wound dressing may, in combination with a stabilizer and/or a barrier disc, also be attached to the said stabilizer and barrier disc in several different ways. The wound dressing may have a premade opening where the stabilizer and/or barrier disc is to be inserted. In some instances the opening may allow for the stabilizer and/or barrier disc to be inserted to a limited degree into the wound dressing. One example is to make a “bag” of low adherent wound contact layer. Inside this there may be another “bag” made of foam and the rigid disc may then be inserted into this “bag”. In other applications the wound dressing may be attached to the exterior of the stabilizer and/or barrier disc without enveloping the same. For instance, the barrier disc may be dressed on the one side with a wound dressing while the other may not be covered with and wound dressing. In some applications, the dressed part of the barrier disc or stabilizer may be facing the tissue of the wound while in some applications the barrier disc or stabilizer may be directly interfering with the wound tissue without any wound dressing present. Additionally, the wound dressing may be attached to the stabilizer and/or barrier disc by any ways know by a skilled person, for example by welding, stapling, sewing and/or using glue or Velcro.

The wound dressing may be made as an integral part of the stabilizer and/or barrier disc. This may be manufactured in any way know by a skilled person: For example, the material of the stabilizer or barrier disc may be manipulated into an open structure pressure transduction material. This may be done by polymerization of the material in the case of polystyrene foam, by extrusion in the case of polyethylene foam or blowing in the case of polyurethane foam. When the stabilizer and/or barrier disc is mad as an integral part the device may also be manufactured using a combination of Injection molding and reaction injection molding in one or several shots to produce an inner core which would correspond to the stabilizer and barrier disc while the outer layer of the device may be open structure to allow pressure transduction.

Such wound dressings are often soft sheets or other soft structures. The wound dressing may be of any suitable material, such as a sponge; a foam such as a polyurethane foam, a polyethylene foam, or a silicone foam; a textile such as rayon, silk, viscose, antimicrobial gauze, absorbant cotton or cotton gauze; cellulose; cellulose ethyl sulphonate with silver; acrylic fibre; polyacrylate fibre; a hydrocolloid; absorbable syntetic polyester; gelatin; glycerin; collagen; pectin; guar gum; sodium alginate; calcium alginate; vinyl acetat; poly-glucosamin; poly-acetylglokosamin; dialkyl carbamoyl chloride; ester acid; polypropelene; resorbable lactidecaprolactone film and/or mesh; collagen matrix; woven polyamide fibres; bioabsorbable PGA:TMC copolymer fibre; multilayered or perforated plastic film; a perforated or open cell teflon sheet/structure; sulphonate with silver, polyurethane film, polyurethane center silver sulferdiazine, nanocrystalline silver coated polyurethane film, acryllic fibre, polyester fim, silicone, polyacrylate fibre, polyaminde tricote net, nylon, polyethylene and/or absorbable syntetic polyester.

The wound dressing may be lined with one or more layers of “low adherence wound contact layer”, in order to hinder ingrowth of the wound bed into the dressing and thereby facilitate the removal of the wound dressing when the therapy is terminated or the dressing is changed, which will lessen the pain upon dressing removal, and also hinder residuals from the dressing getting stuck in the wound. The wound contact layer may be manufactured so that it covers the whole or parts of the wound dressing or rigid barrier disc, it may also be placed so that it covers the underlying tissue or organ structures in the wound and/or over the wound bed.

The term “low adherence wound contact layer” relates to any contact layer known to the skilled person. Such wound dressings are often sheets or other soft structures made of any suitable material. Examples of wound contact layers are perforated plastic film; a membrane of perforated polymeric film, or a textile, such as an open weave cloth, which may be soaked in soft paraffin and/or chlorhexidine, a gauze, a paraffin gauze, or a silicon dressing, a hydrocolloid dressing, a calcium alginate dressing, a perforated or open cell teflon sheet/structure. A wound contact layer may also comprise one or more of the materials given as examples above for the wound dressing. The wound dressing may be of an open pore structure or perforated material. In cases where pressure is not to be transduced onto the wound bed, such structures or sheets may also be non-perforated or solid.

The expression above that the stabilizer is for “stabilization and/or maintenance of a drainage capacity of a wound dressing, i.e. the capability of the wound dressing to drain a wound, means the following:

The stabilizer stabilises and reinforces the wound edges. It keeps the wound tissues separated and hinders these from collapsing when the negative pressure is applied. If the wound collapses pressure transduced maybe lessened or obstructed. The stabilizer thereby maintains the pressure transduction to, and fluid transportation from, deeper parts of the wound.

Another way to facilitate drainage form the wound is to have channels running inside or around the stabilizer, in which pressure can be transduced and fluid removed. At least some channels, may be in form of grooves or slits in the surface of the stabilizer.

Another way of explaining how the stabilizer may facilitate pressure transduction and fluid transportation is as follows: In many situations, when a wound dressing is used in NPWT, the negative pressure results in a compression of the wound dressing in such a way that the wound dressing collapse and the drainage capacity or transportation ability of the wound dressing is impaired. However, the stabilizer may not only stabilise the wound edges, but also the wound dressing when this is placed underneath or around the stabilizer. It holds the wound dressing expanded, hindering it from contracting and obstructing pressure transduction and fluid transportation, following the application of negative pressure. The stabilizer reinforces the wound dressing and keeps the structure of the wound dressing open thereby maintaining its open structure properties and facilitating negative pressure transduction and would fluid removal and thus maintenance of the drainage capacity or transportation ability of the wound dressing after and during application of the negative pressure used in the NPWT. By using the stabilizer according to the present invention, it is possible to obtain a drainage capacity or transportation ability of a wound dressing that remains the same as prior to application of the negative pressure or that is somewhat reduced initially when the pressure is applied but then stabilized at an acceptable level that enables drainage of excess fluid from the wound. This advantage is obtained, at least partly, by stabilization, or maintenance at an acceptable level, of the surface of the wound dressing and/or of the contact surface between the wound dressing and the stabilizer of the barrier disc, i.e. of the drainage surface.

This also leads to a more evenly distributed negative pressure to the wound bed when the stabilizer is used in combination with a barrier disc or a wound dressing, compared to when a similar barrier disc or wound dressing is used without a stabilizer. It may, at least partly, also be explained by the fact that the stabilizer or the combination of the stabilizer and the barrier disc results in an evenly distributed pressure over the surface of the wound dressing. The wound dressing will thus allow pressure transduction to the wound and fluid removal from the wound. Stabilization and/or maintenance of a drainage capacity of a wound dressing will lead to facilitated drainage from a wound dressing or barrier disc, and the expression “facilitating drainage” thus has the same meaning, as explained above for “stabilization and/or maintenance of a drainage capacity”. The fact that drainage is facilitated, which is equal to the fact that the drainage capacity of a wound dressing is stabilized and/or maintained, means in the context of the present invention that drainage will occur speedier than with currently used methods, which in turn means that any infections will be held in a shorter time period and that the total time needed for treatment of the patient will be shortened.

It is possible to use two or more stabilizers according to the invention to enable or facilitate a more efficient NPWT. Whether or not it is possible and/or desirable to use more than one stabilizer will depend on the wound geography. Larger wounds might need more stabilization, or the need for stabilization might vary within the wound. The use of more than one stabilizer will in this context achieve better positioning and stabilization of the barrier disc and/or wound dressing(s). The use of more than one stabilizer may also allow for the physician to use one stabilizer for injection of irrigation fluids through the channel system of that specific stabilizer while an additional stabilizer or more then one additional stabilizers may be used to deliver a negative pressure to the wound. Different stabilizers may also supply different pressures to different compartments in the wound which both can direct irrigation fluids and achieve different therapeutic effects from the use of different pressure zones in the wound bed.

A stabilizer may also be combined from two or more stabilizer, i.e. a stabilizer may be a built from “brick structure” where several stabilizers may be connected to each other to better facilitate the therapy. For instance, the length of body specific wounds such as the sternum wound will depend on the age of the patient where a child has a sternum that is substantially smaller than an adult. A stabilizer may therefore need to be smaller—length wise—when treating a child compared to an adult. Similarly, an obese patient will need a stabilizer which is vertically larger to be able to reach above the subcutaneous tissue of the patient compared to a skinny or normal patient. The skilled person understands that there might be several situations where the stabilizer may need to be customized or have the capability of being arranged in different combinations to be able to achieve the desired therapeutic results. It may be so that a stabilizer may be made from a material which the physician may conform to any shape he finds suitable to fit the purpose of the ridge in a specific application. In certain wounds the physician may want to stabilize the upper wound edges from contracting when the negative pressure is applied while the physician which the sides of the deeper parts of the wound to contract more to allow the wound the heal from the bottom and up. In this situation the stabilizer may preferably be conical where the stabilizer has a larger circumference at the upper part of the stabilizer than at the bottom.

The skilled person realizes that the choice of material, size and shape for the wound dressing will influence drainage capacity. For example, when a foam wound dressing is attached to a stabilizer or to a barrier disc, the stabilizer and/or disc will hinder collaps/deformation of the wound dressing in the plane where it is attached to the stabilizer or barrier disc, while it will not prevent collaps/deformation in the opposite plane. For example, if the wound drssing is attached to a flat barrier disc which is presented horizontally the barrier disc will hinder the deformation of the wound dressing in the lateral plane, while it will not prevent deformation or collaps in the vertical plane. In order to withstand the vertical deformation to a satisfying degree, the skilled persion will realise that the wound dressing will have to have a certain thickness or strength to withstand the forces of deformation. One example of this is that, when a sternotomy wound is treated at −120 mmHg, if the wound dressing is formed by a polyurethane foam with open cell structure of 400-600 μm, a 3 mm thich foam will collaps vertically when arranged on a stabilizer or barrier disc, and drainage through the said foam will not occur in the horisontal direction. On the other hand, a foam with a thickness of at least around 8 to 14 mm will retain its properties of pressure transduction and fluidtransport in the lateral plane. Other wounds may need different specifications of the wound dressing in order to maintain the capacity to deliver negative pressure and allow the transportation of fluid in the plane of the wound dressign. Fore example, when an abdominal wound, that has a larger area, is treated at a lower negative pressure, e.g. −60 mmHg, a less thick wound dressign may be preferable.

Another example is in a neurosurgical wound that probably benefit from treatment at an even lower negative pressure, e.g. −20 to −40 mmHg, an even thinner wound dressign could be used.

The physical properies of the material used for wound dressing will also influence the size and thickness needed to maintain drainage and pressure transmissiont of he wound.

Furthermore, the choice of material, size and shape for the stabilizer will influence the drainage capacity. For example, a stabilizer formed by an open cell structure will not hinder the potential collaps of a wound dressing consisting of an open cell material. It will however prevent partial collaps of the said wound dressing in the plane where the wound dressing is attached to the stabilizer. Thus, the wound dressing will still at least partially collaps in the opposite direction.

The stabilizer may be arranged to protrude substantially perpendicularly to the wound dressing or barrier disc. This is advantageous in many types of wounds, such as sternotomy wounds, in which symmetry of the healing of the wound is beneficial.

According to an embodiment, the stabilizer comprises a ridge arranged to protrude from the surface of the wound dressing or barrier disc. A ridge may be given an elongated shape suitable for use in, e.g., a sternotomy wound.

The stabilizer may comprise a bulge arranged to protrude from the surface of the wound dressing or barrier disc. A bulge may be given a rounded shape suitable for use in, e.g., an abdominal wound.

In an embodiment, the stabilizer is arranged to protrude from the surface of the wound dressing or barrier disc into an opening of a wound in which the wound dressing or barrier disc is used.

The stabilizer may be provided with one, two, three or several channels extending from at least one surface of the stabilizer to at least one other surface of the stabilizer. The channels may improve or simplify drainage. Instead of draining fluids from the wound through a wound dressing, the fluids may be drained through the channels. Further, the channels may be employed for treating different portions of the wound using different pressures. Attaching tubes for drainage is simplified when using channels in the stabilizer, and rubbing against the skin of the patient may be avoided. Facilitated drainage is beneficial, e.g., for draining the pleura after a sternotomy, but also from deep down in other wounds. An effective drainage reduces the risks of fluids collecting in the wound.

The channels may also or alternatively enable delivery of fluids to the wound. A single channel or a set of channels may be used to deliver a fluid, such as a solution, that promotes wound healing to any part or compartment of the wound while a different set of channels may provide a negative pressure at the same time. Depending on the location of the exit point of the delivery system for wound healing fluid and the location of the negative pressure source, the wound healing fluid can be directed to a precise location in the wound site and thereafter the fluid will move towards the negative pressure source. It is also possible to use the same channels for delivery of fluid to the wound and to provide negative pressure to the wound, but this is then not done simultaneously. Fluid may be introduced to the wound in order to rinse the wound, to apply moist or other woudn ehaling factors or analgesic agents. The fluid may be either an inert fluid, e.g. saline, or contain any active factor known to the skilled person. These may for example be used to treat infection, stimulate growth of tissue or provide local anaestetics. Examples of such active factors are interleukines, growth factors, clorhexedine (chlorhexedine gluconate, CHG) derivated, PHMB, intercellular and intracellular signaling molecules VEGF, PDGF, silver, MMPs, starch, kollagen or beta-glukans, non-steroid antiinflamatory agens (NSAID), steroids, local anestetics (suchs as xylocain, mercain cinkokain, or tetrakain) acetylic asid and opoids.

After having delivered a fluid to the wound or a part or compartment of a wound, the fluid may also be evacuated by the channel system in the stabilizor. The channels in the stabilizor may thus be used for rinsing the wound. Fluid is introduced and then sucked away during the negative pressure therapy. The fluid may be introduced at a negative, positive or atmospheric pressure. The channels may aslo be used to intruduce or suck away different gases, such as oxygen, air or nitrogen containing gases. This may be done i.a. to oxygenate wound tissue or to alter the wound bed blood perfusion

According to some embodiments the stabilizer is more or less permanently attached to the barrier disc or the wound dressing. The attachment of the stabilizer to the barrier disc or the wound dressing may be performed in any way known to a person skilled in the art. One way of achieving this is to use an intermediary substance which will bond the components to each other, such as glue or welding. Thus, according to some embodiments, the stabilizer is attached to the barrier disc or wound dressing by means of welding. This is a simple and secure way of fixedly attaching the stabilizer to the barrier disc or wound dressing. Another way of achieving this is to attach the stabilizer to the barrier disc or wound dressing by means of suturing or sewing or any other procedure where an additional material is used to attach the stabilizer to the rigid barrier or wound dressing. The stabilizor may be welded onto the barrier disc or the wound dressing. The stabilizer may be formed as an integral part of the barrier disc.

According to some embodiments the stabilizer is removably attached to the barrier disc or the wound dressing, and to achieve this, an attachment device may be used. The attachment device can be any device that attaches the stabilizer to a barrier disc or a wound dressing, that are known to a person skilled in the art. The stabilizer may comprises an attachment device arranged to attach the stabilizer to the wound dressing or barrier disc. In this manner, the stabilizer may be fixedly or detachably attached to the wound dressing or barrier disc. In some embodiments, the attachement device is a snap-on device. Another example of attachment devices that may be used is a device wherein the parts that are attached to each other, i.e. the stabilizer to a barrier disc or wound dressing, may be attached by means of 3-dimensional structure wherein the stabilizer may be the positive or negative mirror of the 3-dimenstional structure of the attachment device connected to the barrier disc or wound dressing so that the two will lock into each other like a hand in a glove or a key in a lock, or a threaded bolt in threaded hole, or spline solution. The stabilizer may also me attached to a barrier disc or wound dressing by means of a hook and loop attachment device, such as Velcro.

The stabilizer may also be close to the disc without any direct attachment that binds the parts together.

The above-mentioned objects are also achieved, in full or at least in part, by a barrier disc or a wound dressing or for use in negative pressure wound therapy, comprising a stabilizer as discussed above. The stabilizer makes it possible to stabilize the position of the wound dressing or barrier disc in the wound. Further, placement of the wound dressing or barrier disc is simplified, since the person placing the wound dressing or barrier disc may observe the protruding stabilizer in order to verify the position of the wound dressing or barrier disc on which the stabilizer is arranged.

The above-mentioned objects are also achieved, in full or at least in part, by a method for controlling the position of a wound dressing or barrier disc in negative pressure wound therapy, wherein a stabilizer is attached to the wound dressing or barrier disc, said stabilizer extending from the wound dressing or barrier disc into an opening of a wound in which the wound dressing or barrier disc is placed. This makes it possible to easily and securely controt the position of the wound dressing or barrier disc.

The above-mentioned objects are also achieved, in full or at least in part, by a method for facilitating drainage from a wound dressing or barrier disc in negative pressure wound therapy, wherein a stabilizer is attached to the wound dressing or barrier disc, said stabilizer extending from the wound dressing or barrier disc into an opening of a wound in which the wound dressing or barrier disc is placed and comprising channels extending from at least one surface of the stabilizer to at least one other surface of the stabilizer. Drainage may in this way be performed through the channels instead of through a separate sponge. Drainage tubes may be connected directly to the stabilizer channels or the stabilizer channels may emerge from the stabilizer in such a way that an ordinary drainage tube (e.g., TRAC pad or Jackson-Pratt drain) may be connected to or inserted in the dressings, and evacuate fluid through the wound fillers and bandage dressings, in the same manner as during conventional NPWT.

In a variant the method further comprises applying a first negative pressure to a first portion of the wound through a first set of channels in the stabilizer, and applying a second negative pressure to a bandage dressing applied on either side of the stabilizer or through a second set of channels in the stabilizer, said first and second negative pressures being differentiated. In this manner, different portions of the wound may be treated using different pressures. For instance, in an abdominal wound a lesser negative pressure may be used on the sensitive underlying tissues, such as the spleen, intestines, and liver, while a greater negative pressure may be used on the large muscle groups in order to avoid a so called frozen abdomen. Differentiated pressures are also useful in some neurosurgery applications. The pressure differentiation may also involve more than two different pressures.

Other objectives, features and advantages of the present invention will appear from the following detailed disclosure, from the attached claims, as well as from the drawings. It is noted that the invention relates to all possible combinations of features.

Generally, all terms used in the claims are to be interpreted according to their ordinary meaning in the technical field, unless explicitly defined otherwise herein. All references to “a/an/the [element, device, component, means, step, etc.]” are to be interpreted openly as referring to at least one instance of said element, device, component, means, step, etc., unless explicitly stated otherwise. The steps of any method disclosed herein do not have to be performed in the exact order disclosed, unless explicitly stated.

### BRIEF DESCRIPTION OF THE DRAWINGS

The invention will now be described more in detail in the following with reference to the attached drawings, wherein

FIG. 1 shows a perspective view of an embodiment of invention stabilizer and barrier disc seen from below,

FIG. 2 shows a perspective view of the embodiment of the stabilizer according to FIG. 1 having an alternative barrier disc applied, seen from below,

FIG. 3 shows a perspective view of another embodiment of invention stabilizer and barrier disc seen from below,

FIG. 4 shows the embodiment according to FIG. 3 in use at the treatment of an abdominal incision, and

FIG. 5 shows the embodiment according to FIG. 1 in use at the treatment of a sternotomy incision.

FIG. 6a is a highly schematic perspective view of a variation of the shape of a stabilizer.

FIGS. 6b and c are highly schematic cross-sectional views, longitudinally and transversely, respectively, of the stabilizer of FIG. 6a.

FIG. 7 is a highly schematic perspective view of another variation of the shape of a stabilizer.

FIG. 8 is a highly schematic perspective view of yet another variation of the shape of a stabilizer.

FIGS. 9 and 10 schematically show variations of channels in a stabilizer with hidden parts shown in dashed lines.

FIG. 11 shows an embodiment of a stabilizer attached to a wound dressing.

FIGS. 12a-c show how a stabilizer may be attached to a barrier disc using a groove.

FIGS. 13a-d show how a stabilizer may be attached to a barrier disc using a snap-in connection.

FIG. 14 a-c shows a wound dressing shaped as a sleeve (FIG. 14a), into which a barrier disc in arrangement with a stabilizer (FIG. 14b) is introduced (FIG. 14c). FIG. 15 shows a stabilizer attached to a wound dressing consisting of a foam.

FIG. 16 shows a stabilizer attached to a soft wound dressing.

FIG. 17 shows an alternatively shaped stabilizer attached to a soft wound dressing.

FIG. 18 shows an alternative of the embodiment according, wherein the wound dressing comprises a low adherence layer.

FIG. 19 shows an assemby of a barrier disc 11 and a stabilizer 1, wherein the channels in stabilizer are in form of grooves or slits 5″ in the surface of the stabilizer.

FIG. 20 shows an embodiment of the device according to the invention in use at the treatment of an abdominal wound.

### DETAILED DESCRIPTION OF PREFERRED EMBODIMENTS OF THE INVENTION

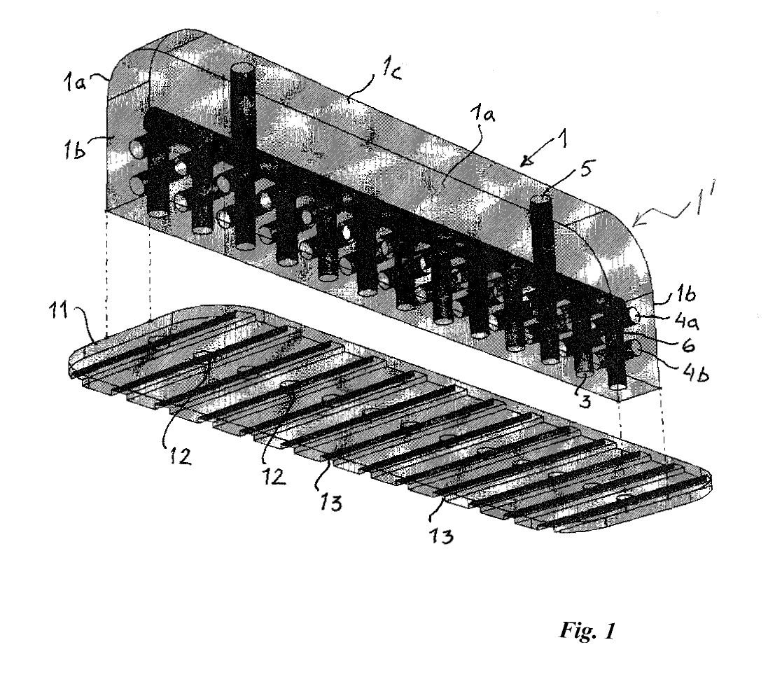

FIG. 1 shows an embodiment of a stabilizer 1 for use in negative pressure wound therapy. The stabilizer 1 generally has a rectangular body having two larger sidewalls 1a, two end walls 1b, a back wall 1c, and a bottom wall 1d, the body being provided with a longitudinally extending (horizontally extending in the figure) channel 2 connecting a series of drainage channels 3 extending vertically. The drainage channels 3 open in the bottom wall 1d. Further, the body 1 is provided with two rows of smaller, shorter channels 4a and 4b passing through said drainage channels 3 extending from one sidewall 1a to the other sidewall 1a. The smaller channels 4a and 4b thus pass from one side to the other of the stabilizer 1. Extending vertically from the longitudinally extending channel 2, two exit channels 5 are provided. The exit channels 5 each have an opening in the back wall 1c.

Thus, the longitudinally extending channel 2 interconnects all channels 2, 3, 4 and 5 forming a drainage network providing for open connections in all walls except the end walls 1b.

The openings of the exit channels 5 in the back wall 1c can be adapted to receive a catheter or channel adaptor (not shown), which channel or catheter is to be connected to a NPWT equipment. Such an adaptor can have a Luer-Slip standard.

The stabilizer 1 is in the embodiment of FIG. 1 connected to a barrier disc 11 provided with a series of perforations 12 in line with the openings of the drainage channels 3. When attached to the barrier disc 11, the stabilizer 1 forms part of the barrier disc 11. The stabilizer 1 may be said to form a ridge 1′ protruding from the barrier disc 11. The barrier disc 11 is further provided with a series of laterally extending slots 13 providing for a transport of fluid drained into the perforated area. These slots 13 open inwardly at the perforation openings 12.

The stabilizer 1 can be detachably connected to the barrier disc 11 by means of a snap-in function, welded to the barrier disc 11, or integrally produced with the barrier disc 11.

The stabilizer 1 may be made in a transparent or at least translucent material to allow inspection of the different channels in an easy way.

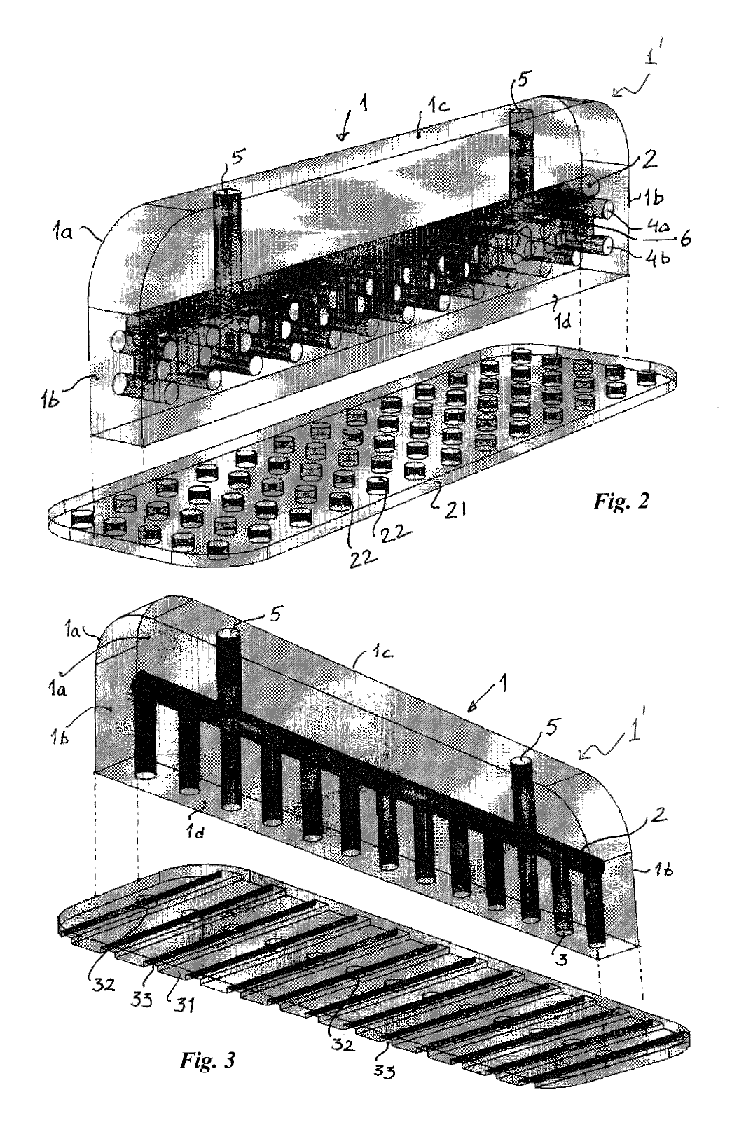

FIG. 2 shows an alternative design of the stabilizer 1. In this figure, the same numerals have been given to same parts as in FIG. 1. Thus, 1 generally denotes a substantially rectangular body having two larger sidewalls 1a, two end walls 1b, a back wall 1c, and a bottom wall 1d, the body being provided with a longitudinally extending (horizontally extending in the figure) channel 2. A series of drainage channels 6 extending vertically interconnect with two rows of smaller, shorter channels 4a and 4b passing through said drainage channels 6, which shorter channels 4a and 4b extend from one sidewall 1a to the other sidewall 1a. The smaller channels 4a and 4b thus pass from one side to the other of the body. Extending vertically from the longitudinally extending channel 2, two exit channels 5 are provided. The exit channels 5 open in the back wall 1c.

Thus the longitudinally extending channel 2 interconnects all channels 2, 6, 4 and 5 forming a drainage network providing for open connections in all walls except the sidewalls 1a and the end walls 1b and the bottom wall 1d.

The openings of the exit channels 5 in the back wall 1c can be adapted to receive a catheter or tube adaptor (not shown), which tube or catheter is to be connected to a NPWT equipment. Such an adaptor can have a Luer standard.

The stabilizer 1 is in the embodiment of FIG. 2 connected to a barrier disc 21 provided with a series of perforations 22. When attached to the barrier disc 21, the stabilizer 1 forms part of the barrier disc 21. The stabilizer 1 may be said to form a ridge 1′ protruding from the barrier disc 21.

The stabilizer 1 can be detachably connected to the barrier disc 21 by means of a snap-in function, welded to the barrier disc 21, or integrally produced with the barrier disc 21.

The stabilizer 1 is preferably made in a transparent or at least translucent material to allow inspection of the different channels in an easy way.

FIG. 3 shows a further alternative design of the stabilizer 1. In this figure the same numerals have been given to same parts as in FIG. 1. Thus, 1 generally denotes a substantially rectangular body having two larger sidewalls 1a, two end walls 1b, a back wall 1c, and a bottom wall 1d, the body being provided with a longitudinally extending (horizontally extending in the figure) channel 2 connecting a series of drainage channels 3 extending vertically. The drainage channels 3 opens in the bottom wall 1d. The smaller shorter channels 4a and 4b present in the designs of FIG. 1 and FIG. 2 are thus not included in this design. Extending vertically from the longitudinally extending channel 2, two exit channels 5 are provided. The exit channels 5 open in the back wall 1c.

Thus, the longitudinally extending channel 2 interconnects all channels 2, 3, and 5 forming a drainage network providing for open connections in all walls except the end walls 1b.

The openings of the exit channels 5 in the back wall 1c can be adapted to receive a catheter or tube adaptor (not shown), which tube or catheter is to be connected to a NPWT equipment. Such an adaptor can have a Luer standard.

The stabilizer 1 is in the embodiment of FIG. 3 connected to a barrier disc 31 provided with a series of perforations 32 in line with the openings of the drainage channels 3. Thus, when attached to the barrier disc 31, the stabilizer 1 forms part of the barrier disc 31. The stabilizer 1 may be said to form a ridge 1′ protruding from the barrier disc 31. The barrier disc 31 is further provided with a series of laterally extending slots 33 providing for a transport of fluid drained into the perforated area. These slots 33 open inwardly at the perforation openings.

The stabilizer 1 can be detachably connected to the barrier disc 31 by means of a snap-in function, welded to the barrier disc 31, or integrally produced with the barrier disc 31.

The stabilizer 1 is preferably made in a transparent or at least translucent material to allow inspection of the different channels in an easy way.

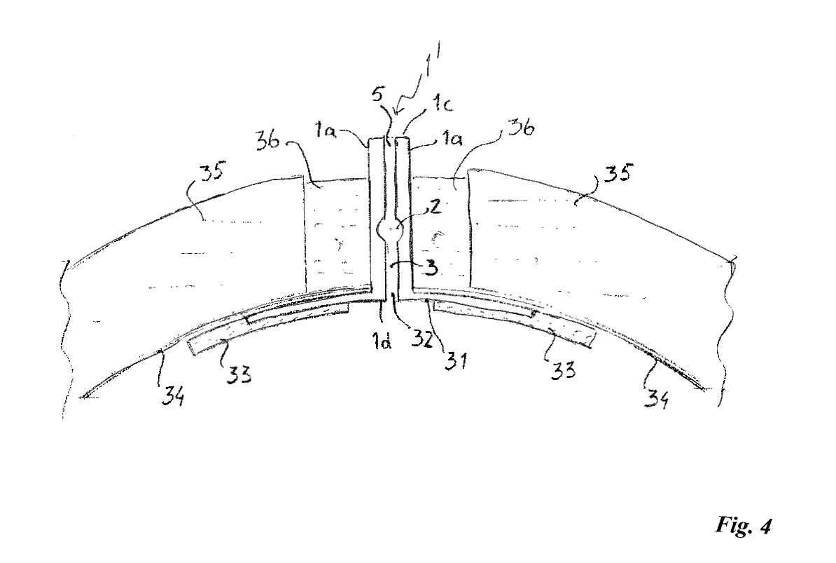

In FIG. 4 it is shown the function of the device according to FIG. 3 when treating an abdominal incision. 31 denotes a barrier disc and the stabilizer 1 is shown attached to the barrier disc 31. On either lateral side of the barrier disc 31 there is a foam sheet 33 extended over the abdominal tissues and organs. The foam sheet 33 is made of a foam material having an open structure to allow a drained fluid to be transported in towards the perforations of the barrier disc and the ridge 1. Further, the foam sheet is covered by a gas impermeable polymer sheet 34, which polymer sheet 34 is attached hermetically to the top, upper side of the barrier disc 31. The abdominal muscle groups 35 are placed above the barrier disc 31 on either side of the stabilizer 1. Between the stabilizer 1, or ridge 1′, and the muscle groups a further foam structure 36 is placed.

A gas impermeable polymer sheet (not shown) is attached above the whole stabilizer 1, the foam structures 36 and parts of the muscle groups in such a way that a hermetic seal is created, in the way as commonly used and practiced at NPWT.

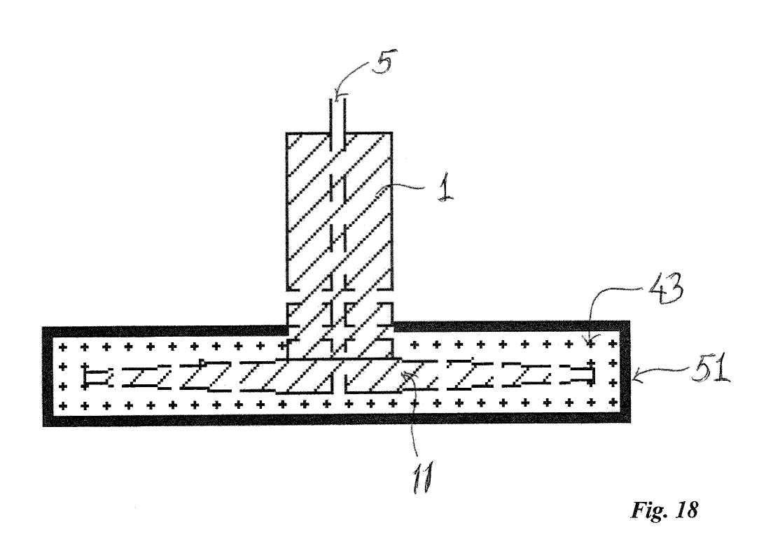

Furthermore, the outside of the wound dressing 43, i.e. the side of the wound dressing 43 that is in contact with the wound when the device comprising a stabilize 1 and a barrier disc 11 with at least one channel 5 is in use, may, at least partly, be covered with a low adherence layer 51, as shown in FIG. 18.

A negative pressure, such as at the level of −30 mmHg, is transferred through the channels 2, 3 and 5 in the stabilizer 1 by connecting the stabilizer 1 to a vacuum pump (not shown) via the openings of the channels 5. On the other hand, two tubes (not shown) are inserted into the foam structure 36, which tubes are connected to a second vacuum source providing a negative pressure of −150 to −200 mmHg. The polymer sheet 34 prevents the negative pressure (−150 to −200 mmHg) from acting on the abdominal cavity. However, the very low negative pressure, less than −150 mmHg, and down to −200 mmHg or even more negative, will draw the muscle groups towards the ridge 1, whereby a frozen abdomen is prevented, i.e., a status where the muscle groups cannot heal together but a space between them becomes chronic. Such a differentiation of the pressures used may also be used with more than two different wound areas treated each with a different pressure. This compartmentalization of the wound may be made in a depth direction, or vertical direction, as well as in a horizontal direction in the wound.

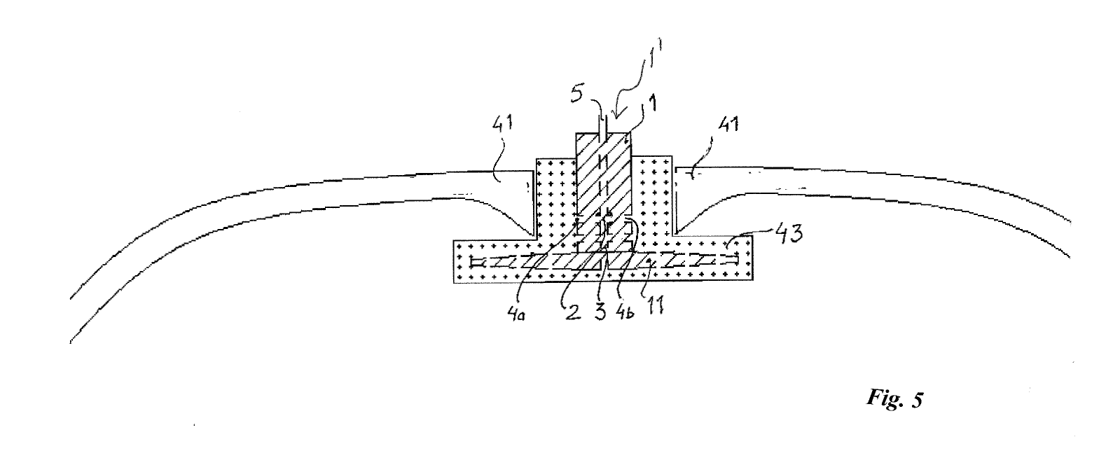

In FIG. 5 it is shown the function of the device according to FIG. 1 when treating a sternotomy incision. The sternal bone is denoted 41. In between the divided sternal bone 41 a stabilizer 1, or ridge 1′, with a connected barrier disc 11 is inserted. On all sides of the stabilizer 1 and the barrier disc 11 there is a foam sheet 43 to cover tissues and organs. The foam sheet 43 is made of a foam material having an open structure to allow a drained fluid to be transported in towards the perforations of the barrier disc 11 and the stabilizer 1. A negative pressure is applied via the channels 5, 2, 3 and 4a and 4b.

A gas impermeable polymer sheet (not shown) is attached above the whole stabilizer 1, the foam structures 43 and parts of the sternal bone in such a way that a hermetic seal is created, in the way as commonly used and practiced at NPWT.

Tests made using only a wound dressing constituted by foam in the cavity between the sternum parts to which the NPWT tubing was connected revealed that when applying the negative pressure the foam was compressed in such a way that its transporting ability was highly decreased. The foam merely collapsed.

However, when applying the foam to the barrier disc, as shown in FIG. 4, or having it enveloping the barrier disc as in FIG. 5 application of the negative pressure will result in no or only minor such compression and the foam maintains, or substantially maintains its drainage capacity or transporting ability. The stabilizer or the stabilizer in combination with the barrier disc will act as a stabilizer of the foam preventing the same from collapsing thereby reducing its drainage capacity or transporting ability. Thus the barrier disc will not only work as a protecting shield to the underlying tissue but will also ensure the transporting capacity of the foam attached thereto. Thus the barrier disc cooperates with the wound dressing to maintain the transporting ability of the latter.

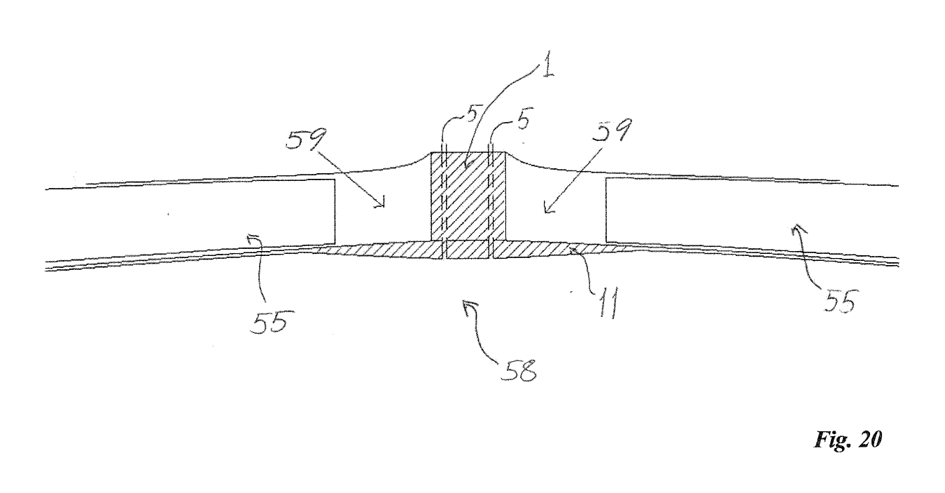

In FIG. 20 it is shown how an embodiment of the device according to the invention comprising a in use at the treatment of an abdominal wound. The abdominal wall is denoted 55. In an opening in the abdominal wall a stabilizer 1 with a connected barrier disc 11 is inserted. A negative pressure is applied via channels 5, resulting in a low negative pressure zone 58 one part of the wound, and a high negative pressure zone 59 in another part of the wound.

Preferably, during NPWT the wound dressing shall cover the part of the stabilizer or barrier disc positioned closest to the organ or underlying tissue and also at least part of the edges of the stabilizer or barrier disc, in order to make sure the drainage surface of the wound dressing is maintained when the pressure is applied during NPWT, thus stabilizing and/or maintaining the drainage capacity of the wound draining.

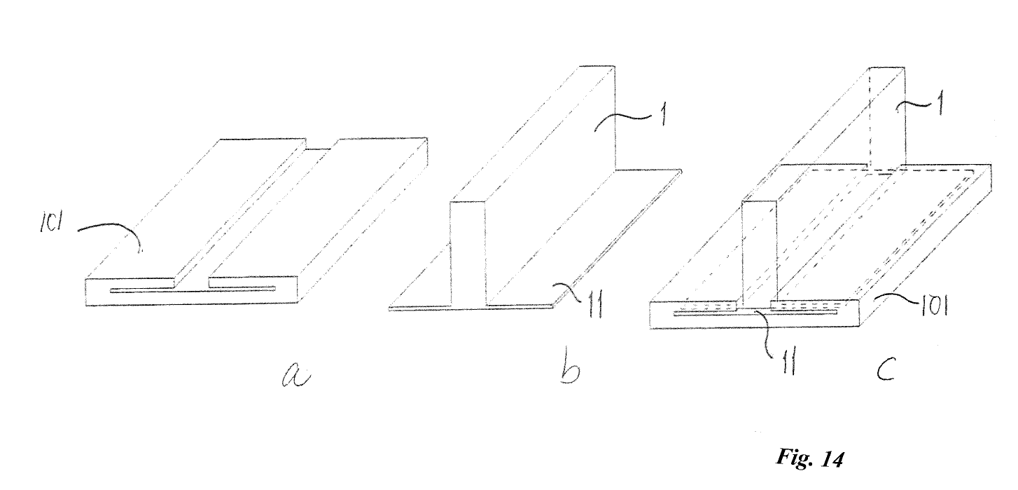

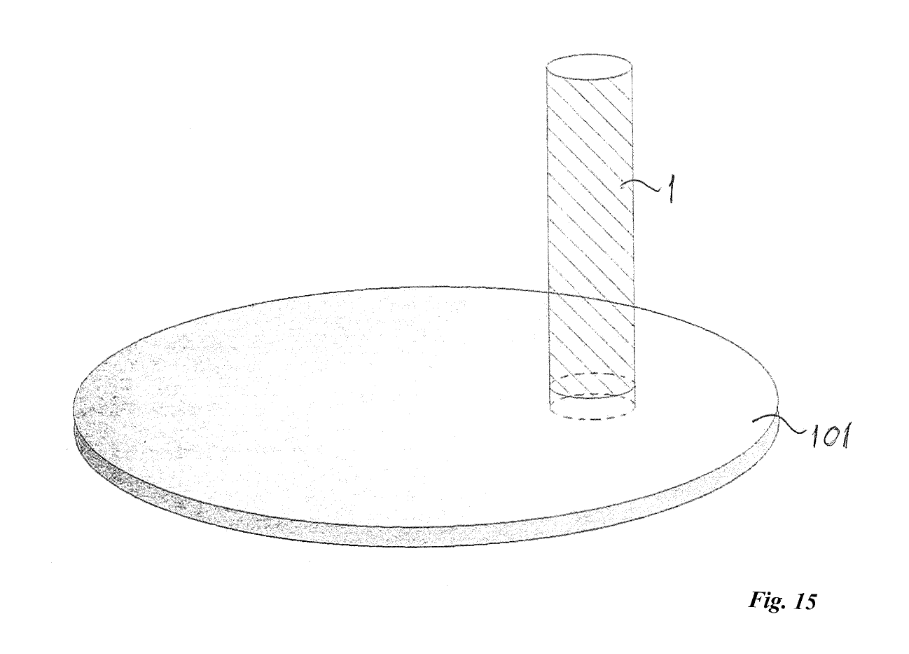

In some embodiments of the invention, the wound dressing, such as a foam, may be shaped as a sleeve, a sheath or a cover, such as shown in FIG. 14a, into which at least part of the stabilizer or barrier disc is introduced, as shown in FIG. 14c. The wound dressing may also consist of a foam, gauze or any other mesh or open structure pressure transduction material, optionally lined as mentioned below, in which a slit or a cut has been provided into which at least part of the stabilizer or barrier disc is introduced.

The skilled person realizes that the shape of the wound dressing in combination with the shape of the stabilizer and/or a barrier disc may take an infinite number of shapes as the shape of wounds may take almost any shape. The wound dressing may, in combination with a stabilizer and/or a barrier disc, also be attached to the said stabilizer and barrier disc in several different ways. The wound dressing may have a premade opening where the stabilizer and/or barrier disc is to be inserted. In some instances the opening may allow for the stabilizer and/or barrier disc to be inserted to a limited degree into the wound dressing. One example is to make a “bag” of low adherent wound contact layer. Inside this there may be another “bag” made of foam and the rigid disc may then be inserted into this. In other applications the wound dressing may be attached to the exterior of the stabilizer and/or barrier disc without enveloping the same. For instance, the barrier disc may be dressed on the one side with a wound dressing while the other may not be covered with and wound dressing. In some applications, the dressed part of the barrier disc or stabilizer may be facing the tissue of the wound while in some applications the barrier disc or stabilizer may be directly interfering with the wound tissue without any wound dressing present. Additionally, the wound dressing may be attached to the stabilizer and/or barrier disc by any ways know by a skilled person, for example by welding, stapling, sewing and/or using glue or Velcro.

The wound dressing may be made as an integral part of the stabilizer and/or barrier disc. This may be manufactured in any way know by a skilled person: For example, the material of the stabilizer or barrier disc may be manipulated into an open structure pressure transduction material. This may be done by polymerization of the material in the case of polystyrene foam, by extrusion in the case of polyethylene foam or blowing in the case of polyurethane foam. When the stabilizer and/or barrier disc is mad as an integral part the device may also be manufactured using a combination of injection molding and reaction injection molding in one or several shots to produce an inner core which would correspond to the stabilizer and barrier disc while the outer layer of the device may be open structure to allow pressure transduction.

In some embodiments the whole, or almost the whole, stabilizer optionally in combination with a barrier disc or at least the whole or almost whole part of the stabilizer and possibly also barrier disc, that are inside a wound during use, may be embedded in wound dressing, such as foam.

In the embodiments discussed in the four preceding passages, the shape of the barrier disc, or the shape of the stabilizer itself if no barrier disc is used, holds the wound dressing, such as foam, in place laterally even when the pressure is applied during use in the NPWT, thus increasing the drainage properties of the wound dressing during NPWT compared to when no stabilizer and/or barrier disc is used.

In some embodiments of the present invention, the wound dressing comprises an open pore/cell structure foam.

In some embodiments of the present invention, the wound dressing comprises a woven material.

In some embodiments of the present invention, the wound dressing comprises a non-woven material.

In some embodiments of the present invention, the wound dressing comprises a open structire material.

In some embodiments of the present invention, the wound dressing is, at least partly, lined with a low adherence wound contact layer. Examples of materials for such wound contact layers are given earlier in this text.

It should be noted that any pressure used at NPWT can be used in connection with the present ridge. Thus, negative pressures between −10 mmHg to −200 mmHg can be used.

The design of the barrier disc is not crucial but can take any form applicable, the only requirement is that the barrier disc used protects the organs to be protected. The particular design will depend on the site of use. In this context, it shall be noted that the barrier disc used together with the stabilizer according to the invention may take several forms and material structures. Thus it may be, for example, flat, shaped like an arc in any one or more directions, oval, circular, quadratic or rectangular. Likewise, it may be rigid, semirigid or flexible with or without flexible edges. The shape and material structure of the barrier disc may be varied with regards to the wound to be treated.

Examples of barrier discs are shown in WO 2007/123451 and WO 2009/142598.

Similarly, the design of the stabilizer is not crucial. For instance, for use in treating a sternotomy wound, the stabilizer or ridge is, when seen from above, preferably elongated, with a width that is several times smaller than the length of the ridge. On the other hand, when treating an abdominal wound, the ridge may in many instances advantageously have a shape as seen from above which is closer to a square or circle. In order to improve safety and comfort, any corners of the shape are preferably rounded.

The term “ridge” should in this context be construed broadly, and not necessarily as resembling an elongated, geological formation. A “ridge” may in this context be any formation protruding above the surface of the barrier disc. Seen from above, the ridge may have a generally elongated, rectangular shape or a generally square or circular shape, which could also be referred to as a bulge. The shape of the ridge need not necessarily be symmetrical or uniform, but could be, e.g., curved or tapering.

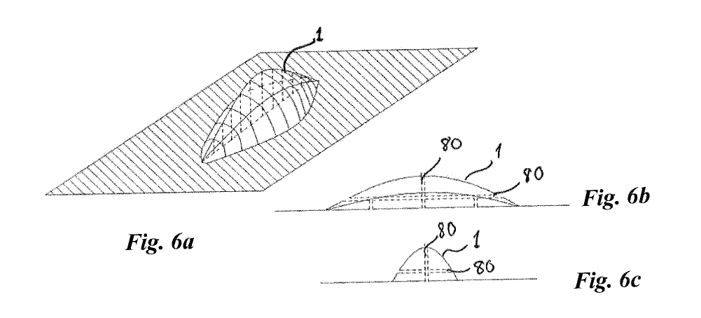

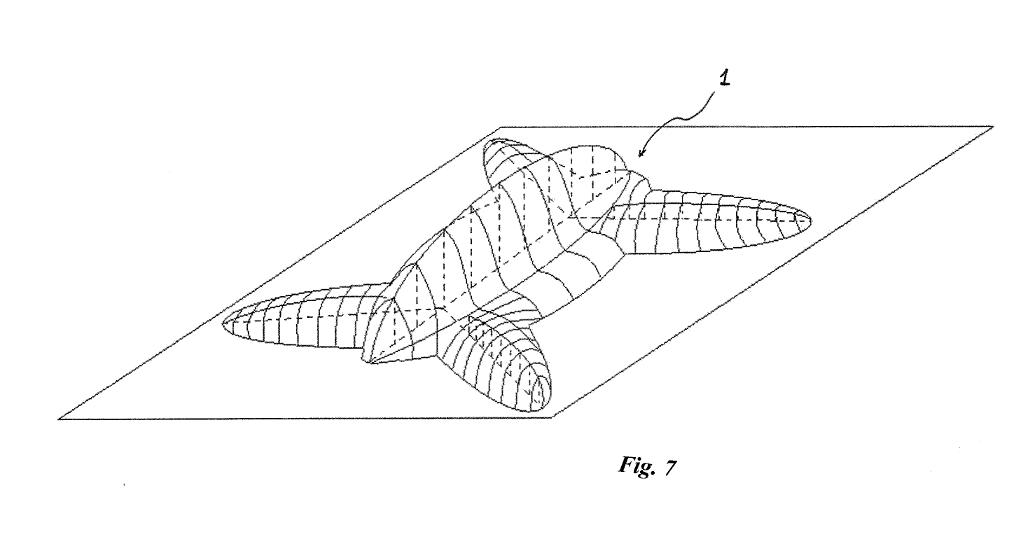

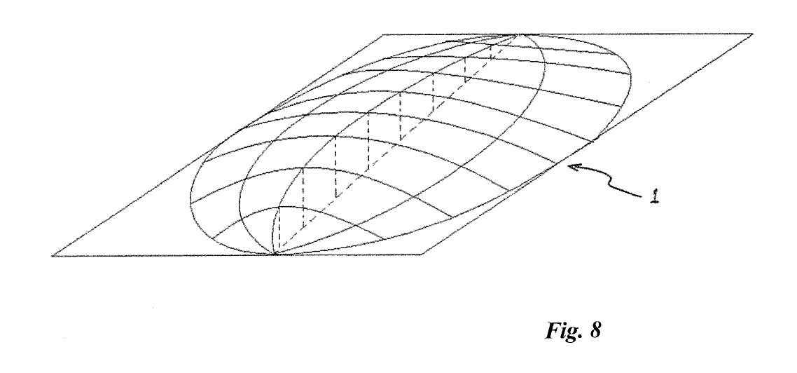

FIGS. 6-8 very schematically show some examples of variations of the shape of the stabilizer 1. In FIG. 6a, the stabilizer 1 has a shape resembling the hull of a boat. FIG. 6b shows schematically in a longitudinal cross-section how channels 110 in the stabilizer may extend. FIG. 6c is a corresponding transverse cross section. FIG. 7 depicts a turtle-like shape of the stabilizer, with a “body” having a “shell” and four “legs”. FIG. 8 shows an oval, slightly bevelled stabilizer 1.

The stabilizer need not necessarily extend along the entire length of the barrier disc. Further, possibly, more than one stabilizer could be arranged on one and the same barrier disc.

The material of the stabilizer may be the same as or different from the material of the barrier disc in connection with which it is to be used. Any material that have the physical properties to maintain stabilisation of the wound and/or maintain fluid transportation orm the wound, that are known to a person skilled in the art. It may be a biocompatible polymer or co-polymer material, such as a clinical silicone material, or a polylactic polymer or copolymer. Other examples of materials are polytetrafluoroethylene, polyester, polypropylene, acrylonitrile/butadiene/styrene, esters and ethers, nylon, cellulose acetate propiconate, butyrate, polychlorotrifluoroethylene, polyvinyl fluoride, polyvinylidene fluoride, ethylene-tetrafluoroethylene, fluorinated ethylene propylene, polyacetals polymethylmethacrylate, polyacrylonitrile, polyacrylate, polycyanoacrylate, aliphatic and amorphous grades, aromatic polyimide/polyimide, polycarbonate, and/or polyethylene (LDPE, LLDPE, HDPE, UHMPE, UHMWPE), viscose, cellulose; cellulose ethyl sulphonate with silver; acrylic fibre; polyacrylate fibre; absorbable syntetic polyester; pectin; vinyl acetat; poly-glucosamin; poly-acetylglokosamin; collagen matrix; polyamide fibres; bioabsorbable PGA:TMC copolymer fibre; acryllic fibre, polyacrylate fibre, and/or titanium or any other suitable metal or alloy.

If the stabilizer is manufactured as a separate part, different materials may be chosen for the stabilizer and the barrier disc, allowing optimization of material properties for each part. If, on the other hand, the stabilizer is manufactured as an integral part of the barrier disc, it will in many cases be more cost efficient to use the same material for the entire stabilizer—barrier disc assembly.

The material should provide sufficient mechanical stability that the stabilizer can perform its stabilizing function, and should be sufficiently flexible to allow such movement of surrounding tissues and bones as is required for the comfort of the patient. For use in treatment of wounds including bones, such as sternotomy, the material needs to provide greater mechanical stability than for use in treatment of wounds including only soft tissues, such as abdominal wounds. On the other hand, for use in wounds in soft tissues, the material needs to provide greater flexibility than for use in wounds including bones, such that the stabilizer may better conform to the inner organs of the patient.

The stabilizer may have different shapes. It may further be solid or comprise channels.

In embodiments having channels, the material of the stabilizer needs to provide sufficient mechanical stability to prevent the channels from collapsing at the pressures employed, e.g., −10 to −200 mmHg.

When arranged on the barrier disc and placed in a wound, the stabilizer may protrude outside the wound. Alternatively, an upper edge of the stabilizer may be flush with the skin of the patient or be placed slightly below the skin.

In embodiments having channels, the channels may extend from any one surface of the stabilizer to any other surface of the stabilizer, depending on how the channels are to be used. In the embodiments shown in the drawings, there are no channel openings in the end walls 1b, but it would also be possible to have channel openings in the end walls 1b, e.g., for drainage. Instead of in the back wall 1c, the openings of the exit channels 5 could be formed in the side walls 1a, preferably close to the back wall 1c for ease of access.

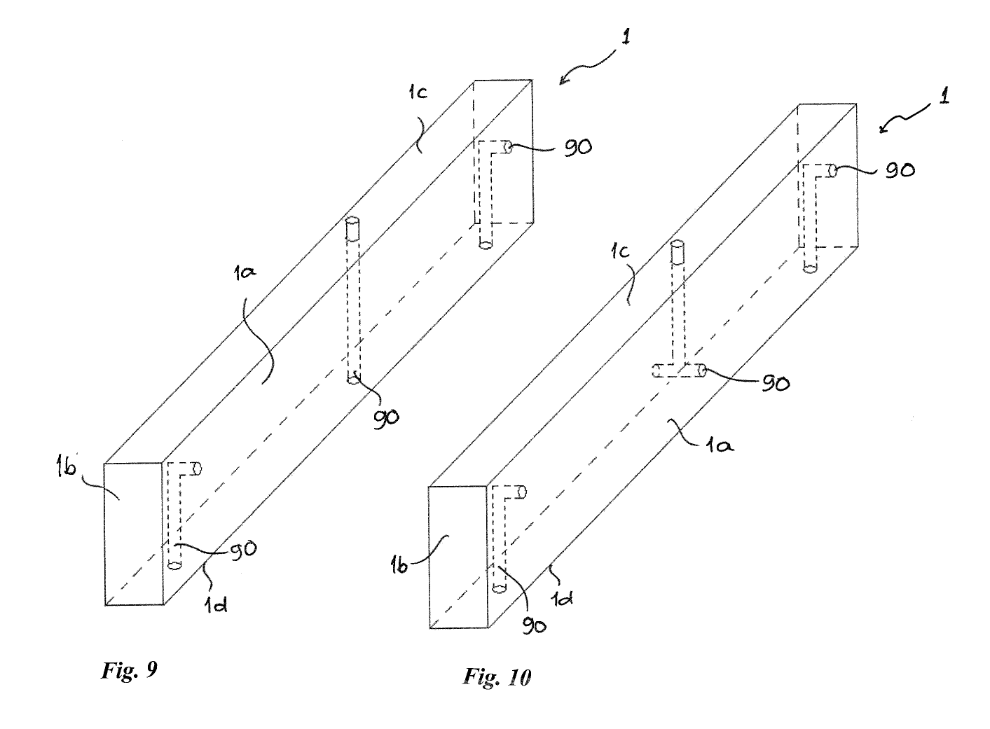

FIGS. 9 and 10 schematically show examples of how channels may be arranged in the stabilizer 1. As may be seen from FIG. 9, channels 90 may extend from the bottom wall 1d to a side wall 1a, or from the bottom wall 1d to the back wall 1c. FIG. 10 shows that channels may extend from one or both of the side walls 1a to the back wall 1c. The configurations of the channels may be combined freely depending on the drainage that is to be achieved.

For use in other treatments of wounds, except negative pressure wound therapy, it will not always be necessary to incorporate channels in the stabilizer.

The stabilizer 1 may be attached to the barrier disc in any suitable way, either permanently or detachably. For instance, the stabilizer may be glued, welded or sewn onto the barrier disc. The stabilizer may be provided with an attachment device, such as a snap on device for attachment to the barrier disc. The stabilizer may be provided with a groove which is slidable onto a profile on the barrier disc. Alternatively, such a profile may be provided on the stabilizer and the groove formed in the barrier disc.

FIGS. 12a-c show how a stabilizer 1 may be attached to a barrier disc 61 using a groove 62 on the barrier disc 61 into which a profile 63 on the barrier disc 61 is slidable.

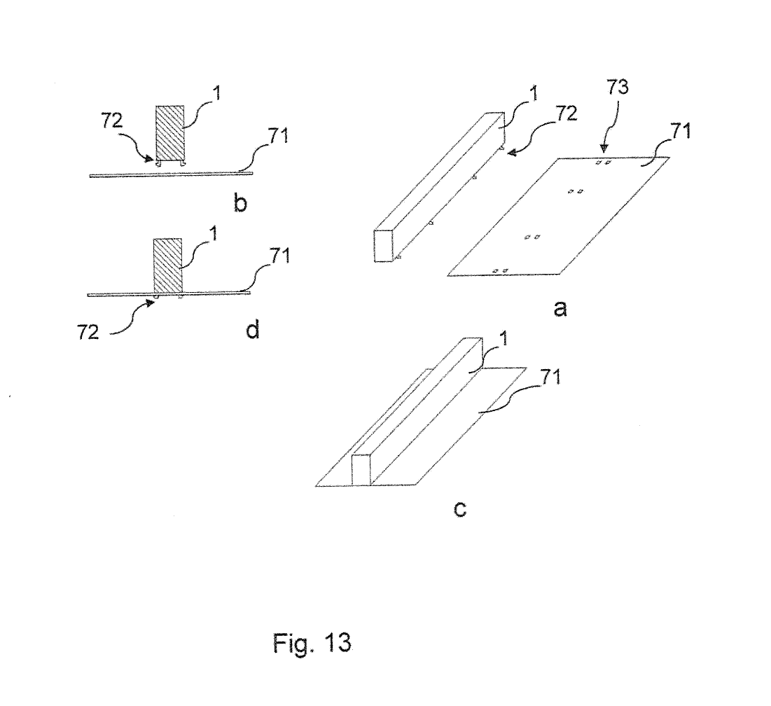

FIGS. 13a-d show an alternative way of attaching the stabilizer to the barrier disc. Here, the stabilizer 1 has snap-in connectors 72, which may snap into apertures 73 in the barrier disc 71.

FIG. 14 a-c show an alternative way of attaching a wound dressing to a barrier disc attached to the stabilizer. The wound dressing is shaped as a sleeve, as shown in FIG. 14a, into which a barrier disc in arrangement with a stabilizer, as shown schematically in FIG. 14b may be introduced, as shown in FIG. 14c.

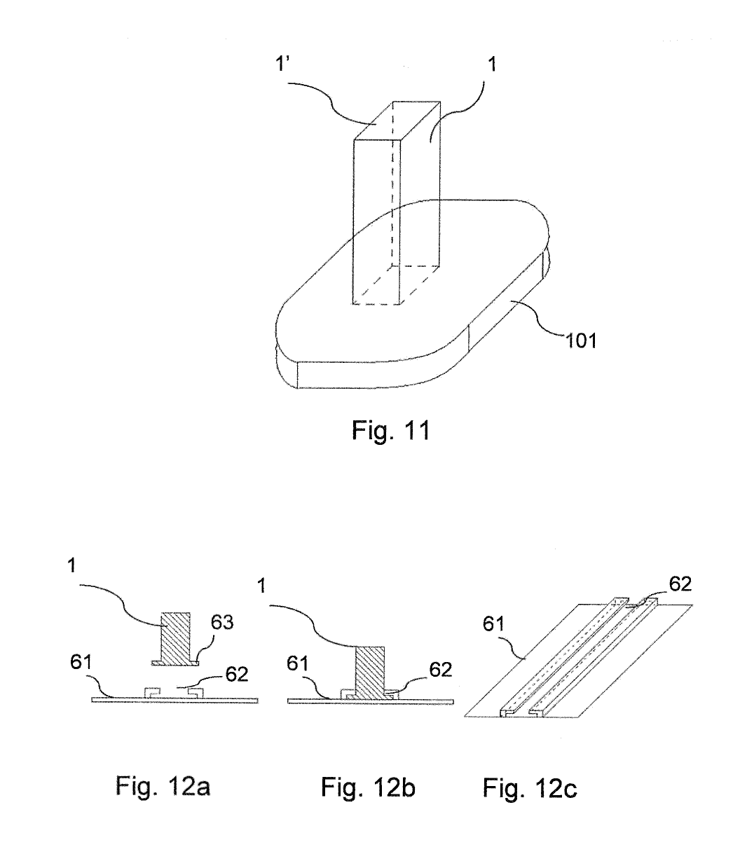

FIG. 11 shows a stabilizer 1 attached to a wound dressing 101 instead of a barrier disc. The wound dressing 101 will generally be softer than the barrier discs discussed above. The stabilizer 1 may be attached to the wound dressing in any suitable way, e.g., by sewing, gluing, or laminating. Alternatively, the stabilizer 1 may be used in conjunction with a with a wound dressing without fixedly attaching the stabilizer 1 to the wound dressing 101, by placing the stabilizer 1 on top of the wound dressing 101. Many of the advantages described above in relation to the use of the stabilizer in conjunction with a barrier disc may also be achieved when using the stabilizer in conjunction with a wound dressing.

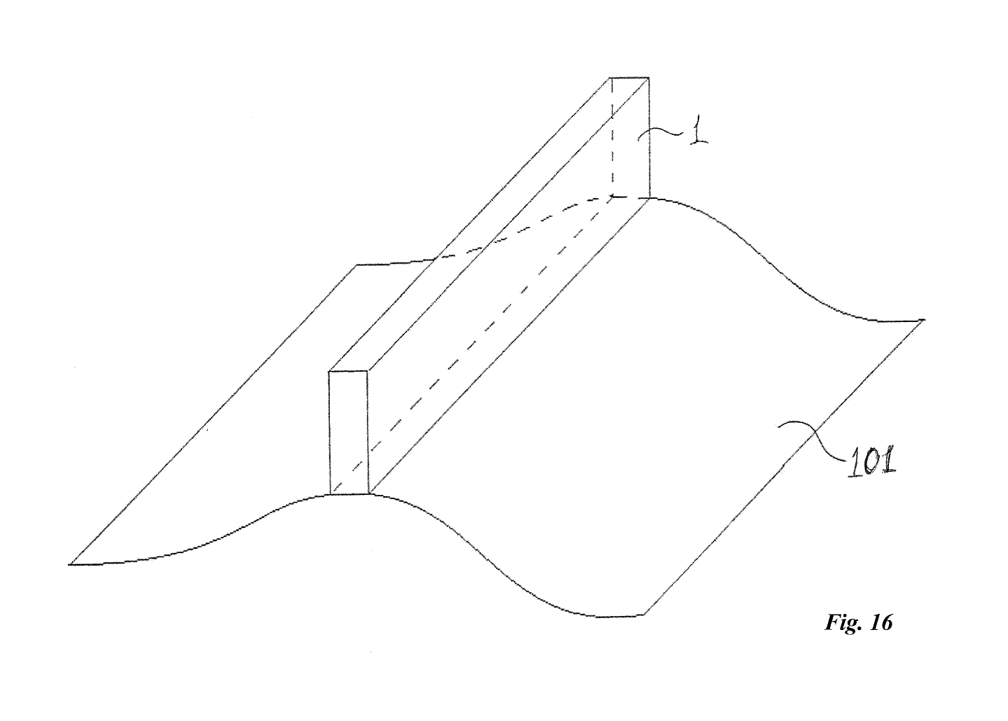

FIGS. 15 and 16 schematically show examples of different types of wound dressing; a foam as shown in FIG. 15 and a soft wound dressing as shown in FIG. 16.

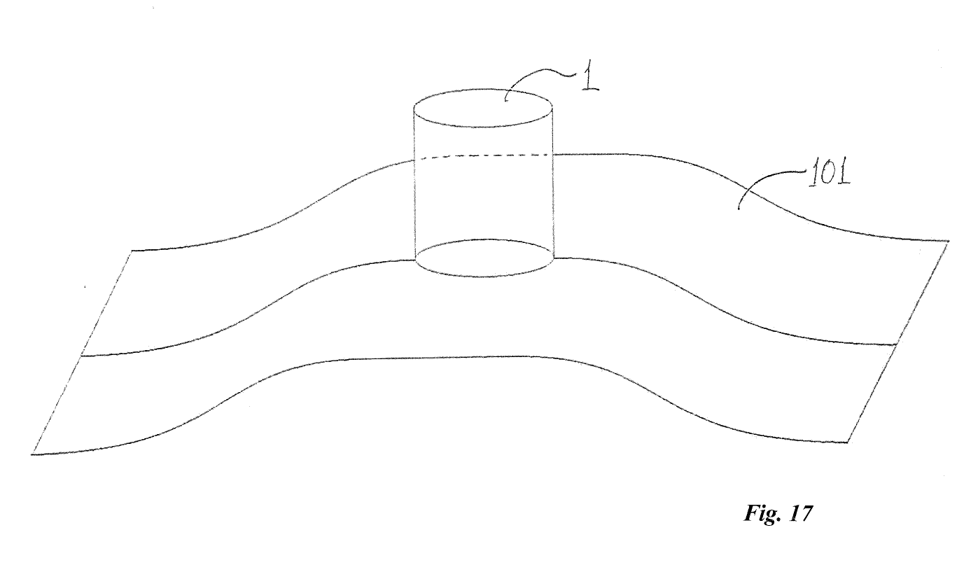

FIG. 17 schematically shows examples of how channels may be arranged in the stabilizer 1. As may be seen from FIG. 9, channels 90 may extend.

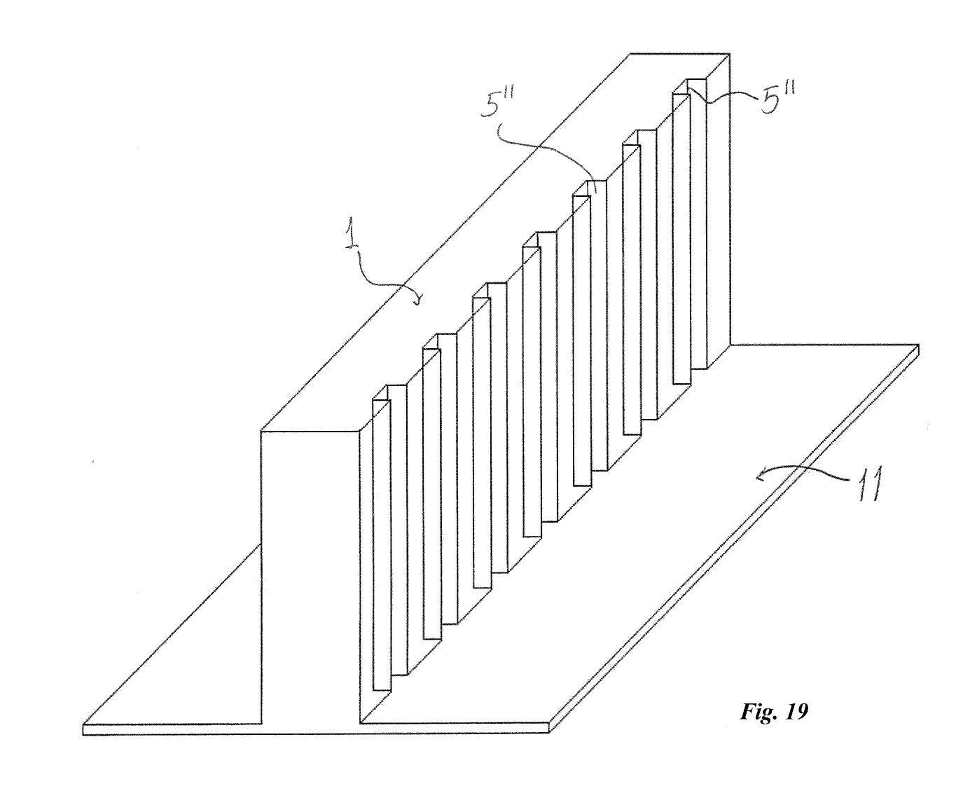

FIG. 19 schematically shows an assembly of a barrier disc and a stabilizer wherein the channels in stabilizer are in form of grooves or slits in the surface of the stabilizer.

In the embodiments discussed above, the stabilizer is used attached to a barrier disc or wound dressing. However, the stabilizer may also be used on its own, still providing the possibilities of stabilizing wound edges, facilitating drainage, and differentiating pressures.

### REFERENCES

Agarwal J P, Ogilvie M, Wu L C, Lohman R F, Gottlieb L J, Franczyk M, Song D H. (2005) Vacuum-assisted closure for sternal wounds: a first-line therapeutic management approach. Plast Reconstr Surg. 116(4):1035-40; discussion 1041-3
Anesäter E, Borgquist O, Robertsson P, Torbrand C, Roupé K M, Ingemansson R, Lindstedt S, Malmsjö M. “The use of a rigid disc to protect exposed structures in wounds treated with negative pressure wound therapy—effects on wound bed pressure and microvascular blood flow”. In press Wound Rep Regen.
Anesäter E, Roupé M, Robertsson P, Borgquist O, Torbrand C, Ingemansson R, Lindstedt S, Malmsjö M. “The influence on wound contraction and fluid evacuation of a rigid disc inserted to protect exposed organs during negative pressure wound therapy”. Int Wound J 2011 August; 8(4):393-9.
Anesäter et al (2012)E, Borgquist O, Torbrand C, Roupe K M, Ingemansson R, Lindstedt S, et al. A Rigid Disc for Protection of Exposed Blood Vessels During Negative Pressure Wound Therapy. Surg Innov. 2012.
Argenta L C, Morykwas M J. (1997) Vacuum-assisted closure: a new method for wound control and treatment: clinical experience. Ann Plast Surg. June; 38(6):563-76;
Baillot R, Cloutier D, Montalin L, Côté L, Lellouche F, Houde C, Gaudreau G, Voisine P. (2009) Impact of deep sternal wound infection management with vacuum-assisted closure therapy followed by sternal osteosynthesis: a 15-year review of 23499 sternotomies. Eur J Cardiothorac Surg. October 30. [Epub ahead of print]
Bapat V, El-Muttardi N, Young C, Venn G, Roxburgh J. (2008). Experience with Vacuum-assisted closure of sternal wound infections following cardiac surgery and evaluation of chronic complications associated with its use. J Card Surg. 23(3):227-33.
Borger M A, Rao V, Weisel R D, Ivanov J, Cohen G, Scully H E, David T E. (1998) Deep sternal wound infection; risk factors and outcomes. Ann Thorac Surg. 65:1050-6.
Conquest A M, Garofalo J H, Maziarz D M, Mendelson K G, Su Sun Y, Wooden W A, Meadows W M, Nifong W, Chitwood W R. (2003) Hemodynamic effects of the vacuum-assisted closure device on open mediastinal wounds. Surg Res. December; 115(2):209-13.
Cowan K N, Teague L, Sue S C, Mahoney J L. (2005) Vacuum-assisted wound closure of deep sternal infections in high-risk patients after cardiac surgery. Ann Thorac Surg. 80(6):2205-12.
Domkowski P W, Smith M L, Gonyon D L Jr, Drye C, Wooten M K, Levin L S, Wolfe W G. (2003) Evaluation of vacuum-assisted closure in the treatment of poststernotomy mediastinitis. J Thorac Cardiovasc Surg. 126(2):386-90.
Doss M, Martens S, Wood J P, Wolff J D, Baier C, Moritz A (2002) Eur J Cardiothorac Surg. 22(6):934-8. Vacuum-assisted suction drainage versus conventional treatment in the management of poststernotomy osteomyelitis.
Douville E C, Asaph J W, Dworkin R J, Handy J R Jr, Canepa C S, Grunkemeier G L, Wu Y. (2004). Sternal preservation: a better way to treat most sternal wound complications after cardiac surgery. Ann Thorac Surg. 78(5):1659-64.
Ennker I C, Malkoc A, Pietrowski D, Vogt P M, Ennker J, Albert A. (2009) The concept of negative pressure wound therapy (NPWT) after poststernotomy mediastinitis—a single center experience with 54 patients. J Cardiothorac Surg. January 12; 4:5.
Fuchs U, Zittermann A, Stuettgen B, Groening A, Minami K, Koerfer R. (2005). Clinical outcome of patients with deep sternal wound infection managed by vacuum-assisted closure compared to conventional therapy with open packing: a retrospective analysis. Ann Thorac Surg. February; 79(2):526-31
Gustafsson R, Johnsson P, Algotsson L, Blomquist S, Ingemansson R. (2002) Vacuum-assisted closure therapy guided by C-reactive protein level in patients with deep sternal wound infection. J Thorac Cardiovasc Surg. May; 123(5):895-900.
Hersh R E, Jack J M, Dahman M I, Morgan R F, Drake D B. (2001). The vacuum-assisted closure device as a bridge to sternal wound closure. Ann Plast Surg. 46(3):250-4.
Khoynezhad A, Abbas G, Palazzo R S, Graver L M. (2004) Spontaneous right ventricular disruption following treatment of sternal infection. J Card Surg. January-February; 19(1):74-8. Review.
Kofidis T, Emmert M Y, Paeschke H G, Emmert L S, Zhang R, Haverich A. (2009) Long term follow up after minimal invasive direct coronary artery bypass grafting procedure: a multifactorial retrospective analysis at 1000 patient-years. InteractCardiovasc Thorac Surg. September 4. [Epub ahead of print]
Lindstedt S, Ingemansson R, Malmsjö M. (2011a) Sternum wound contraction and distension during negative pressure wound therapy when using a rigid disc to prevent heart and lung rupture. J Cardiothoracic Surg. 2011: 6:42.
Lindstedt S, Ingemansson R, Malmsjö M. (2011 b) A rigid barrier between the heart and sternum protects the heart and lungs against rupture during negative pressure wound therapy. J Cardiothoracic Surg. 2011: 6:90.
Lindstedt S, Malmsjö M, Ingemansson R. (2011c) Effects on draiage of the mediastinum and pleura during negative pressure wound therapy when using a rigid barrier to prevent heart rupture. Int Wound J. 2011; 8(5):454-8.
Lindstedt S, Malmsjö M, Hansson J, Hlebowicz J, Ingemansson R. (2011d) Macroscopic changes during negative pressure wound therapy of the open abdomen using conventional NPWT and NPWT using a protective disc over the intestines. J BMC Surg 2011; 11:10.
Lindstedt S, Ingemansson R, Malmsjö M, (2011e) Haemodynamic effects of negative pressure wound therapy when using a rigid barrier to prevent heart rupture. Int Wound J. 2011; 8(4):385-92.
Lindstedt S, Malmsjö M, Hansson J, Hlebowicz J, and Ingemansson R. (2012b) Pressure transduction and fluid evacuation during conventional negative pressure wound therapy of the open abdomen and NPWT using a protective disc over the intestines. BMC Surg. 2012; 12:4
Lindstedt S, Malmsjö M, Hansson J, Hlebowicz J, Ingemansson R. (2012a) Microvascular blood flow changes in the small intestinal wall during conventional negative pressure wound therapy and negative pressure wound therapy using a protective disc over the intestines in laparostomy. Annals of Surgery 2012; 255(1):171-5.
Lu J C, Grayson A D, Jha P, Srinivasan A K, Fabri B M. (2003) Risk factors for sternal wound infection and mid-term survival following coronary artery bypass surgery. Eur J Cardiothorac Surg. 23:943-9.
Luckraz H, Murphy F, Bryant S, Charman S C, Ritchie A J. (2003) Vacuum-assisted closure as a treatment modality for infections after cardiac surgery. J Thorac Cardiovasc Surg. February; 125(2):301-5.
Malmsjö M, Ingemansson R, Sjögren J. (2007) Mechanisms governing the effects of vacuum-assisted closure in cardiac surgery. Plast Reconstr Surg. October; 120(5):1266-75.
Malmsjö M, Petzina R, Ugander M, Engblom H, Torbrand C, Mokhtari A, Hetzer R, Arheden H, Ingemansson R. (2009) Preventing heart injury during negative pressure wound therapy in cardiac surgery: assessment using real-time magnetic resonance imaging. J Thorac Cardiovasc Surg. 138(3):712-7.
Malmsjö M, Lindstedt S, Ingemansson R. (2011) Effects on heart pumping function when using foam and gauze for negative pressure wound therapy of sternotomy wounds. J Cardiothorac Surg. January 13; 6:5.
Mangram A J, Horan T C, Pearson M L, Silver L C, Jarvis W R. (1999) Guideline for Prevention of Surgical Site Infection, 1999. Centres for Disease Control and Prevention (CDC) Hospital Infection Control Practices Advisory Committee. Am J Infect Control 27:97-132
Morykwas M J, Argenta L C, Shelton-Brown E I, McGuirt W. (1997) Vacuum-assisted closure: a new method for wound control and treatment: animal studies and basic foundation. Ann Plast Surg. June; 38(6):553-62.
Obdeijn M C, de Lange M Y, Lichtendahl D H, et. al. (1999) Vacuum-assisted closure in the treatment of poststernotomy mediastinitis. Ann Thorac Surg 68:2358-2360.
Poncelet A J, Lengele B, Delaere B, Zech F, Glineur D, Funken J C, El Khoury G, Noirhomme P. (2008) Algorithm for primary closure in sternal wound infection: a single institution 10-year experience. Eur J Cardiothorac Surg. 33(2):232-8.
Raja S G, Berg G A. (2007) Should vacuum-assisted closure therapy be routinely used for management of deep sternal wound infection after cardiac surgery? Interact Cardiovasc Thorac Surg. 6(4):523-7. Epub April 20
Ringelman P R, Vander Kolk C A, Cameron D, Baumgartner W A, Manson P N. (1994) Long-term results of flap reconstructions in median sternotomy wound infections. Plast Reconstr Surg. 93:1208-14.
Sartipy U, Lockowandt U, Gäbel J, Jidéus L, Dellgren G. (2006) Cardiac rupture during vacuum-assisted closure therapy. Ann Thorac Surg. September; 82(3):1110-1.
Scholl L, Chang E, Reitz B, Chang J. (2004) Sternal osteomyelitis: use of vacuum-assisted closure device as an adjunct to definitive closure with sternectomy and muscle flap reconstruction. J Card Surg. 19(5):453-61.
Sjögren J, Nilsson J, Gustafsson R, Malmsjö M, Ingemansson R. (2005 a). The impact of vacuum-assisted closure on long-term survival after poststernotomy mediastinitis. Ann Thorac Surg. October; 80(4):1270-5.
Sjögren J, Gustafsson R, Nilsson J, Malmsjö M, Ingemansson R. (2005 b). Clinical outcome after poststernotomy mediastinitis: vacuum-assisted closure versus conventional treatment. Ann Thorac Surg. 79(6):2049-55
Sjögren J, Malmsjö M, Gustafsson R, Ingemansson R. (2006) Poststernotomy mediastinitis: a review of conventional surgical treatments, vacuum-assisted closure therapy and presentation of the Lund University Hospital mediastinitis algorithm. Eur J Cardiothorac Surg. December; 30(6):898-905. Epub 2006 Oct. 23.
Sjögren J, Mokhtari A, Gustafsson R, Malmsjö M, Nilsson J, Ingemansson R. (2008) Vacuum-assisted closure therapy for deep sternal wound infections: the impact of learning curve on survival and predictors for late mortality. Int Wound J 5:216-223.
Yuen J C, Zhou A T, Serafin D, Georgiade G S. (1995) Long-term sequelae following median sternotomy wound infection and flap reconstruction. Ann Plast Surg 35:585-9.

1. A stabilizer for stabilization of the position of a wound dressing and/or barrier disc for use in negative pressure wound therapy, said stabilizer being arranged to protrude from a surface of the wound dressing or barrier disc.

2. A stabilizer according to claim 1, arranged to protrude substantially perpendicularly to the wound dressing or barrier disc.

3. A stabilizer according to claim 1, comprising a ridge arranged to protrude from the surface of the wound dressing or barrier disc.

4. A stabilizer according to claim 1, comprising a bulge arranged to protrude from the surface of the wound dressing or barrier disc.

5. A stabilizer according to claim 1, wherein the stabilizer is arranged to protrude from the surface of the wound dressing or barrier disc into an opening of a wound in which the wound dressing or barrier disc is used.

6. A stabilizer according to claim 1, wherein the stabilizer is provided with channels extending from at least one surface of the stabilizer to at least one other surface of the stabilizer.

7. A stabilizer according to claim 1, further comprising an attachment device arranged to attach the stabilizer to the wound dressing or barrier disc.

8. A stabilizer according to claim 7, wherein the attachment device is a snap-on device.

9. A barrier disc or wound dressing for use in treatment of a wound, comprising a stabilizer according to claim 1.

10. A barrier disc or wound dressing according to claim 9, said barrier disc or wound dressing being arranged for use in negative pressure wound therapy.

11. A barrier disc according to claim 9, wherein the stabilizer is attached to the barrier disc by means of welding.

12. A barrier disc according to claim 9, wherein the stabilizer is an integral part of the barrier disc.

13. A method for controlling the position of a wound dressing or barrier disc in negative pressure wound therapy, wherein a stabilizer is attached to the wound dressing or barrier disc, said stabilizer extending from the wound dressing or barrier disc into an opening of a wound in which the wound dressing or barrier disc is placed.

14. A method for maintaining or facilitating pressure transduction to the wound or drainage from a wound dressing or barrier disc in negative pressure wound treatment, wherein a stabilizer is attached to the wound dressing or barrier disc, said stabilizer extending from the wound dressing or barrier disc into an opening of a wound in which the wound dressing or barrier disc is placed and may comprise channels extending from at least one surface of the stabilizer to at least one other surface of the stabilizer.

15. A method according to claim 14, further comprising applying a first negative pressure to a first portion of the wound through a first set of channels in the stabilizer, and applying a second negative pressure to a bandage dressing applied on either side of the stabilizer, or to a second set of channels in the stabilizer, said first and second negative pressures being differentiated.
